(19) United States
(12) Patent Application Publication
(10) Pub. No.: US 2011/0288377 A1
(43) Pub. Date: Nov. 24, 2011

(54) BIOLOGICAL INFORMATION MEASUREMENT APPARATUS AND METHOD THEREOF

(75) Inventor: Mahoro Anabuki, Yokohama-shi (JP)

(73) Assignee: CANON KABUSHIKI KAISHA, Tokyo (JP)

(21) Appl. No.: 13/109,726

(22) Filed: May 17, 2011

(30) Foreign Application Priority Data
May 21, 2010 (JP) 2010-117665

(51) Int. Cl. A61B 5/00

(52) U.S. Cl. 600/300

(57) ABSTRACT

A biological information measurement apparatus includes an input unit configured to input a captured image of a subject whose biological information is to be measured, a surrounding condition recognition unit configured to recognize surrounding conditions of the subject from the image, and a control unit configured to control a measurement unit, which measures the biological information of the subject, based on the surrounding conditions recognized by the surrounding condition recognition unit.

### BACKGROUND OF THE INVENTION

1. Field of the Invention

The present invention relates to a biological information measurement apparatus and a method thereof.

2. Description of the Related Art

For the purpose of examinations of influenza, metabolic syndromes or the like, and control of lifestyle-related diseases, a vital value such as a body temperature, weight, blood pressure, and heart rate is continuously measured to compare the vital value and a reference value determined beforehand. However, since biological information can be fluctuated due to an external factor such as behavior or clothes of the measurement subject, the biological information needs to be measured by choosing a state in which such an external fluctuation factor does not exist.

However, for example, if a biological information measurement subject is a young child, an elderly person, or the like, it is difficult for the biological information measurement subject by herself/himself to determine such a state. Further, even if the biological information measurement subject can determine the state, when the biological information is continuously measured for a long period of time, it is troublesome to determine the state every time.

To solve such an issue, for example, Japanese Patent No. 3,821,744 discusses a service in which the state of a biological information measurement subject is recognized from the biological information measurement result of the biological information measurement subject to urge the biological information measurement subject to measure the biological information when the state is suitable for measurement.

However, the external fluctuation factor of the biological information is not limited to the state of the biological information measurement subject herself/himself. The measurement subject is affected by an atmospheric temperature, a location, a person being present together, or the like when the biological information is measured. Thus, the biological information may fluctuate.

For example, when the heart rate is measured directly after exercise, the heart rate is naturally increased more than that at a resting period. When weight is measured in a state warmly dressed, the weight is naturally more heavily measured. Further, when a blood pressure is measured at the place where a doctor or a nurse is present in an examination room, a phenomenon in which the blood pressure is measured higher than usual may occur owing to a person. This is known as “white coat hypertension”.

### SUMMARY OF THE INVENTION

The present invention is directed to a biological information measurement apparatus and a method thereof capable of measuring biological information of a biological information measurement subject in consideration of a fluctuation due to surrounding conditions of the biological information measurement subject.

According to an aspect of the present invention, a biological information measurement apparatus includes an input unit configured to input a captured image of a subject whose biological information is to be measured, a surrounding condition recognition unit configured to recognize surrounding conditions of the subject from the image, and a control unit configured to control a measurement unit, which measures the biological information of the subject, based on the surrounding conditions recognized by the surrounding condition recognition unit.

Further features and aspects of the present invention will become apparent from the following detailed description of exemplary embodiments with reference to the attached drawings.

### BRIEF DESCRIPTION OF THE DRAWINGS

The accompanying drawings, which are incorporated in and constitute a part of the specification, illustrate exemplary embodiments, features, and aspects of the invention and, together with the description, serve to explain the principles of the invention.

FIG. 1 is a block diagram illustrating a functional configuration of a biological information processing apparatus according to a first exemplary embodiment.

FIG. 2 is a flowchart illustrating an example of processing to be executed by the biological information processing apparatus according to the first exemplary embodiment.

FIG. 3 is a block diagram illustrating a functional configuration of a biological information processing apparatus according to a second exemplary embodiment.

FIG. 4 is a flowchart illustrating an example of processing to be executed by the biological information processing apparatus according to the second exemplary embodiment.

### DESCRIPTION OF THE EMBODIMENTS

Various exemplary embodiments, features, and aspects of the invention will be described in detail below with reference to the drawings.

A biological information processing apparatus according to the present exemplary embodiment is installed in a space where a biological information measurement subject is acting and recognizes surrounding conditions of the subject of the measurement. Then, while considering the recognition result, biological information of the measurement subject is measured. The biological information in the present exemplary embodiment refers to a so-called vital value. More specifically, the biological information includes a weight, blood pressure, heart rate, pulse, blood sugar level, body temperature, sweating quantity, or the like.

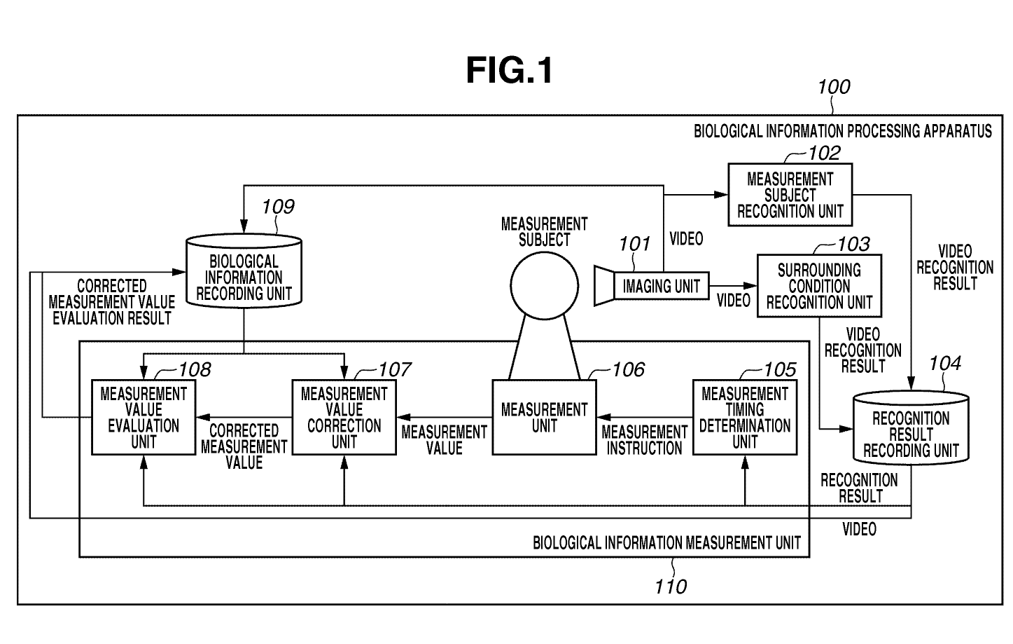

FIG. 1 is a diagram illustrating an outline of a biological information processing apparatus 100 according to the present exemplary embodiment. As illustrated in FIG. 1, the biological information processing apparatus 100 includes an imaging unit 101, a measurement subject recognition unit 102, a surrounding condition recognition unit 103, a recognition result recording unit 104, a biological information measurement unit 110, and a biological information recording unit 109. Similarly, as illustrated in FIG. 1, the biological information measurement unit 110 contained in the biological information processing apparatus 100 includes a measurement timing determination unit 105, a measurement unit 106, a measurement value correction unit 107, and a measurement value evaluation unit 108.

The imaging unit 101 includes a camera and captures an image of an actual space where a measurement subject is acting. The actual space where the measurement subject is acting is, for example, a living room in a house where the measurement subject lives. The imaging unit 101 may be suspended from the ceiling; placed on a floor, a stand, or a television; or incorporated in furniture such as a television, a mirror, a table, and a chair.

The captured video is output to the measurement subject recognition unit 102 and the surrounding condition recognition unit 103. A parameter of a camera operation such as pan, tilt, and zoom may be fixed or changeable. The imaging unit 101 may also include a sensor (e.g., human motion sensor, temperature sensor) to measure a phenomenon in which the state of the actual space, where the measurement subject is acting, is reflected.

The measurement subject recognition unit 102 receives a video from the imaging unit 101 to execute video recognition for the measurement subject. “Video recognition for measurement subject” is first to recognize “whether the measurement subject is included in a video”. Then, when it is recognized that the measurement subject is included, the measurement subject recognition unit 102 recognizes “what sort of expression, posture, movements, and behavior the measurement subject exhibits, what sort of clothes the measurement subject wears, and what the measurement subject possesses”.

For example, the measurement subject recognition unit 102 recognizes “whether the measurement subject is included in a video” by determining whether a region similar to the face image of the measurement subject retained in the measurement subject recognition unit 102 is included in a partial region of the video received from the imaging unit 101. Further, the measurement subject recognition unit 102 may also recognize “whether the measurement subject is included in a video” by determining the presence or absence of a region similar to an image of clothes that the measurement subject has.

The measurement subject recognition unit 102 recognizes expression, posture, movements, behavior, clothes, and possessions of the measurement subject by determining whether the partial region of the video received from the imaging unit 101 is similar to any among image features that represent expression, posture, movements, behavior, clothes, objects, and the like to be retained beforehand. For example, when the partial region of the video received from the imaging unit 101 by which the measurement subject is recognized is similar to the image feature that represents a certain posture, the measurement subject recognition unit 102 recognizes that the measurement subject maintains such the posture.

The measurement subject recognition unit 102 transmits the recognition result to the recognition result recording unit 104 together with the time at which the recognition is executed. Further, the measurement subject recognition unit 102 may also transmit the video to be recognized to the recognition result recording unit 104 together with the recognition result.

The surrounding condition recognition unit 103 receives the video from the imaging unit 101 to execute video recognition for the surrounding conditions of the measurement subject. “Video recognition for surrounding conditions of measurement subject” is first to recognize “whether a person other than the measurement subject is included in a video”.

Then, when it is recognized that a person other than the measurement subject is included, the surrounding condition recognition unit 103 recognizes “what sort of expression, posture, movement, and behavior the person exhibits, what sort of clothes the person wears, what the person possess”, or the like. Further, recognizing “whether an object other than the person is included in the video, and if the object is included, what it is and in what state it is” is also included in “video recognition for surrounding conditions of measurement subject”.

For example, the surrounding condition recognition unit 103 determines “whether a heating appliance such as an air conditioner is included in the video”. Further, the surrounding condition recognition unit 103 determines “in what operation state the heating appliance is working (whether strong warm air is supplied, whether it is not operated, etc.) “. More specifically, the surrounding condition recognition unit 103 determines “whether a heating appliance such as an air conditioner is included in the video” based on whether the image feature of the heating appliance retained inside is detected in the video received from the imaging unit 101.

Then, the surrounding condition recognition unit 103 determines “in what operation state the heating appliance is working” based on whether the image feature (e.g., the image feature when indicator lamp is turned on), which indicates the operation state of the heating appliance, is detected in the video region where the heating appliance is detected. The surrounding condition recognition unit 103 can determine the operation condition (whether strong warm air is supplied, whether it is not operated, etc.) by listing a difference in image features that represent the operation state (position of the recognized indicator lamp and type thereof).

When the imaging unit 101 includes a sensor with which a phenomenon to which the state of the actual space where the measurement subject is acting is reflected, is measured, the surrounding condition recognition unit 103 may also execute “video recognition for surrounding conditions of measurement subject” using the measurement value by the sensor. For example, when a human motion sensor included in the imaging unit 101 detects presence of a person, the surrounding condition recognition unit 103 can complete recognition in shorter time by recognizing only the video region where the detection location of the human motion sensor is included.

The surrounding condition recognition unit 103 transmits the recognition result to the recognition result recording unit 104 together with the time of the recognition. Further, the surrounding condition recognition unit 103 may also transmit the video to be recognized together to the recognition result recording unit 104.

The recognition result recording unit 104 receives the recognition result associated with the recognition time from each of the measurement subject recognition unit 102 and the surrounding condition recognition unit 103, and records thereof. As a result, in the recognition result recording unit 104, the history of the recognition result for the measurement subject and the recognition result for the surrounding conditions of the measurement subject is recorded. The history of the recorded recognition results is referred to by the biological information measurement unit 110. Not only the history of the recognition result but also the video to be recognized may also be recorded in the recognition result recording unit 104.

The biological information measurement unit 110 executes processing concerning measurement of the biological information. More specifically, the biological information measurement unit 110 determines a timing to measure the biological information, measures the biological information, corrects the measurement value of the measured the biological information, and evaluates the measurement value of the biological information. The biological information measurement unit 110 transmits the result that has been subjected to a series of biological information measurement processing, to the biological information recording unit 109.

The measurement timing determination unit 105 determines a timing to measure the biological information of the measurement subject using the history of the recognition result for the measurement subject and the recognition result for the surrounding conditions of the measurement subject. For example, when the recognition result for the measurement subject that the location of the measurement subject in the image is the same during a certain time period and also the posture is not changed, is obtained, the measurement timing determination unit 105 determines that the measurement subject is in a rest state. Then, the measurement timing determination unit 105 determines to measure the biological information at the timing.

Further, for example, when the recognition result for the surrounding conditions is obtained that a heating appliance located around the measurement subject is not long after starting operation, the measurement timing determination unit 105 determines that the body of the measurement subject is not warmed. Then, the measurement timing determination unit 105 does not measure the biological information at the timing.

The measurement timing determination unit 105 executes determination of the measurement timing based on the history of such recognition results. As a method for determining the measurement timing, for example, there is a rule base method in which the timing to measure the biological information is determined at the moment at which the acting state of the measurement subject is determined from the recognition result and the determination result satisfies a certain condition (e.g., acting state, e.g., “rest”, continues five minutes or longer).

Further, the measurement timing determination unit 105 may have a historical pattern of the recognition results and the contents of determination thereto inside as a list to determine whether the timing is a measurement timing according to the list every time referring to the history of the recognition results. When it is determined to be the timing to measure the biological information, the measurement timing determination unit 105 transmits an instruction to measure the biological information to the measurement unit 106.

The measurement unit 106 is a sensor to measure the biological information of the measurement subject. For example, as an example, the measurement unit 106 includes a weight scale, a thermometer, a sphygmomanometer, a heart rate meter, and the like.

The measurement unit 106 may measure the biological information by directly contacting the measurement subject, or may measure without contacting the measurement subject. As an example of the latter, the measurement unit 106 includes a body surface temperature sensor using infrared thermography, an aspirated air detector by using a camera, and the like. Even if it is realized with any sensor, the measurement unit 106 is disposed so as to allow the biological information to be measured when the measurement subject is included in the video captured by the imaging unit 101.

For example, in a case where the measurement unit 106 is a weight scale, when the measurement subject is present in the imaging range of the imaging unit 101, the measurement unit 106 is disposed at the feet thereof. Further, for example, when the measurement unit 106 is a body surface temperature sensor using infrared thermography, the measurement unit 106 is disposed so that the imaging range of the imaging unit 101 and the measuring range of the body surface by the measurement unit 106 are overlapped.

The timing at which the measurement unit 106 measures the biological information is determined by the measurement timing determination unit 105. More specifically, if an instruction to measure the biological information is received from the measurement timing determination unit 105, the measurement unit 106 measures the biological information. The measurement unit 106 transmits the measurement value of the biological information obtained by measurement, to the measurement value correction unit 107.

The measurement value correction unit 107 corrects the measurement value of the biological information of the measurement subject, if necessary, using the history of the recognition result for the measurement subject and the recognition result for the surrounding conditions of the measurement subject. For example, when the recognition result for the measurement subject represents that the measurement subject is warmly dressed, the measurement value correction unit 107 subtracts the weight thereof from the measurement value of the biological information as correction since the warmly dressed weight is added to the weight measured as the biological information.

This “warmly dressed weight” is set to the measurement value correction unit 107, a storage device to which the measurement value correction unit 107 can refer, or the like beforehand for each measurement subject, for example, 2 kg for Mr. A, 1 kg for Ms. B, and the like. Further, the measurement value correction unit 107 may also estimate weight proportional to a height as the warmly dressed weight from the height of the measurement subject estimated from the video captured by the imaging unit 101. Furthermore, the measurement value correction unit 107 may also execute more detail recognition to determine “warmly dressed weight” in such a manner that when it is recognized to be warmly dressed with a coat, “warmly dressed weight” is 2 kg, and when it is recognized to be warmly dressed with a sweater, “warmly dressed weight” is 1 kg, and the like.

Further, for example, when the recognition result for the surrounding conditions represents that a doctor is present near the measurement subject who suffers from white coat hypertension, the measurement value correction unit 107 subtracts an increase in blood pressure due to the white coat hypertension from the measurement value of the biological information as correction.

The measurement value of the biological information corrected by the measurement value correction unit 107 is transmitted to the biological information recording unit 109 via the measurement value evaluation unit 108 and recorded therein. Further, the measurement value correction unit 107 may correct the measurement value of the measurement subject received from the measurement unit 106 by referring to the previously corrected measurement value recorded in the biological information recording unit 109.

For example, with respect to the correction amount of a blood pressure value based on white coat hypertension, the measurement value when previously the white coat hypertension was not developed (when a doctor or nurse was not present near the measurement subject during measurement) is to be recorded for each individual. The measurement value correction unit 107 estimates the correction amount from this value.

The measurement value evaluation unit 108 evaluates the measurement value using the history of the recognition result for the measurement subject and the recognition result for the surrounding conditions of the measurement subject to associate it with a tag that indicates the result. “Evaluation” refers to whether the measurement subject and the surrounding conditions thereof during the measurement of the biological information affect on the measurement value of the biological information. Accordingly, the tag that indicates the evaluation result refers to an influential factor to the measurement value of the biological information.

For example, the body temperature of a human is known to fluctuate in a certain rhythm even in one day (on the morning, it is low and on the evening, it is highest). Thus, associating it with the measurement timing is useful when grasping a long term body temperature fluctuation. Accordingly, when a measurement timing of the biological information is in the early morning and the recognition result for the measurement subject is immediately after awaking, the measurement value evaluation unit 108 evaluates the body temperature measured as the biological information to be the body temperature in the early morning and after awaking and associates an “early morning and also x minutes after awaking” tag with the measurement value of the biological information.

As another example, when while the measurement timing is in the early morning, a sleeping state is not contained in the past six hours of the recognition result, the measurement value evaluation unit 108 evaluates the body temperature measured as the biological information to be the body temperature when the measurement subject has worked through the night and has finished his work, and associate an “after through the night” tag with the measurement value of the biological information.

Similarly, a blood pressure also fluctuates due to mental excitement, sadness, stress, or anxiety. It is useful to record a blood pressure while associating with such conditions for the purpose of more surely grasping a long term blood pressure fluctuation.

Accordingly, for example, when the recognition result for the surrounding conditions represents that a smiling friend is present near the measurement subject, the measurement value evaluation unit 108 evaluates the blood pressure measured as the biological information to be a blood pressure affected by a mild mental state, and associates a “mild” tag with the measurement value of the biological information. At this time, the measurement value evaluation unit 108 may execute evaluation also in view of the measurement value of the biological information itself. In other words, the measurement value evaluation unit 108 associates a “mild” tag with the measurement value of the biological information as it is, if the blood pressure value, the heart rate, and the amount of sweating have a normal value with the similar recognition result obtained.

On the other hand, when the blood pressure value, the heart rate, and the amount of sweating are high, the measurement value evaluation unit 108 may also associate a “not peaceful at heart” tag therewith. Further, the measurement value evaluation unit 108 may also execute evaluation of not only the recognition result and the measurement value of the biological information but also the measurement value of the biological information in view of the audio recognition result and the like.

As another example, when the recognition result for the surrounding conditions indicates that a fiend with an angry face is present near the measurement subject, the measurement value evaluation unit 108 evaluates the blood pressure measured as the biological information to be affected by a state of stress and associates a “stress” tag with the measurement value of the biological information.

Also at this time, the measurement value evaluation unit 108 may execute evaluation also in view of the measurement value of the biological information itself. In other words, when the blood pressure value, the heart rate, and the amount of sweating are high with the similar recognition result obtained, the measurement value evaluation unit 108 associates a “stress” tag with the measurement value of the biological information as it is.

On the other hand, when the blood pressure value, the heart rate, and the amount of sweating have a normal value, the measurement value evaluation unit 108 may also associate an “peaceful at heart” tag therewith. Further, since the recognition result obtained by referring to the recognition result recording unit 104 represents a factor that directly affects the measurement value of the biological information, the measurement value evaluation unit 108 may also employ the recognition result as a tag as it is.

The measurement value of the biological information evaluated by the measurement value evaluation unit 108 and provided with a tag is transmitted to the biological information recording unit 109 as the biological information together with the tag. Further, the measurement value evaluation unit 108 may also evaluate the measurement value of the biological information by referring to the past measurement value of the biological information recorded in the biological information recording unit 109 and associated with a tag.

For example, in a case where relatively many “mild” tags are attached to the blood pressure value measured when a certain person is present together with the measurement subject in the past, the measurement value evaluation unit 108 changes an evaluation standard so that evaluation of the blood pressure value measured when the certain person is present with the measurement subject tends to become “mild” easily, and executes evaluation. In other words, the measurement value evaluation unit 108 changes the upper limit of the blood pressure value which becomes a standard to determine whether to be “mild” based on the past measurement value of the biological information recorded in the biological information recording unit 109 and associated with a tag.

The biological information recording unit 109 receives the measurement value of the biological information associated with a tag from the measurement value evaluation unit 108 to record this inside. The recorded measurement value of the biological information is referred to by the measurement value correction unit 107 and the measurement value evaluation unit 108. Further, the recorded measurement value of the biological information is disclosed to a measurement subject, a doctor, and the like via a display or the like (not illustrated). Furthermore, it is referred to by a system (not illustrated) and used for realizing a biological information service.

When the measurement value of the biological information is recoded, the biological information recording unit 109 may also record a video captured by the imaging unit 101 at the moment the biological information is measured, or before and after the biological information is measured together therewith. The video may be obtained by referring to that recorded in the recognition result recording unit 104 or may directly be obtained from the imaging unit 101.

The configuration concerning the biological information processing apparatus 100 according to the present exemplary embodiment is described above.

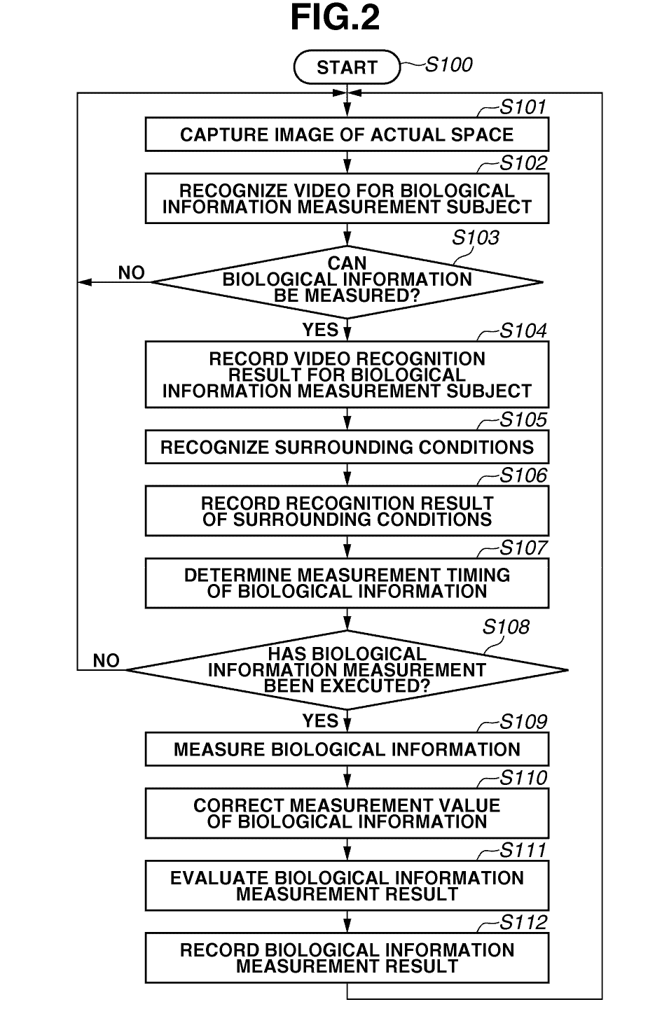

Subsequently, the processing to be executed by the biological information processing apparatus 100 according to the present exemplary embodiment will be described with reference to a flowchart in FIG. 2.

When the processing is started in step S100, first in step S101, the imaging unit 101 captures an image of an actual space where the measurement subject is acting. The imaging unit 101 transmits the captured video to the measurement subject recognition unit 102 and the surrounding condition recognition unit 103. Then, the processing proceeds to step S102.

In step S102, the measurement subject recognition unit 102 executes video recognition processing for the measurement subject to the video transmitted from the imaging unit 101. Through this processing, “whether the measurement subject is included in the video”, and “what expression, posture, movements, and behavior the measurement subject exhibits, what clothes the measurement subject wears, and what the measurement subject has” are recognized.

As a result, for example, the recognition result such as “the measurement subject is absent”, “the measurement subject remains in the same place”, and “the measurement subject is warmly dressed” is obtained. Thereafter, the processing proceeds to step S103.

In step s103, the measurement subject recognition unit 102 determines whether the biological information of the measurement subject can be measured based on the video recognition processing result performed in step S102. In other words, when the measurement unit 106 is an attaching type, the measurement subject recognition unit 102 determines that the biological information can be measured, if the measurement subject is in a range where the imaging unit 101 captures an image.

When the measurement unit 106 is not the attaching type, the measurement subject recognition unit 102 determines that the biological information can be measured, if the measurement subject is in a range of measurement of the biological information. In either of these two cases, if the measurement subject recognition unit 102 determines that the biological information can be measured (YES in step S103), the processing proceeds to step S104. Otherwise (NO in step S103), more particularly, when the measurement subject is not present in the image capturing range or in the biological information measuring range, the processing returns to step S101.

In step S104, the measurement subject recognition unit 102 records the video recognition result for the measurement subject executed in step S102 in the recognition result recording unit 104 together with the recognition time information. Thereafter, the processing proceeds to step S105.

In step S105, the surrounding condition recognition unit 103 executes video recognition processing for the surrounding conditions of the measurement subject to the video transmitted from the imaging unit 101 using a known technique. Through this processing, “whether a person other than the measurement subject is included in the video”, “what expression, posture, movements, and behavior the person exhibits, what sort of clothes the person dresses, and what the person has”, and “whether an object other than a person is included in the video, and if the object is included, what the object is and in what state the object is” are recognized.

As a result, for example, the recognition result such as “the measurement subject is one”, “the measurement subject is present together with a smiling person”, and “a heating appliance is operated” is obtained. Thereafter, the processing proceeds to step S106.

In step S106, the surrounding condition recognition unit 103 records the video recognition result for the surrounding conditions of the measurement subject executed in step S105 in the recognition result recording unit 104 together with the recognition time information. Thereafter, the processing proceeds to step S107.

In step S107, the measurement timing determination unit 105 determines a timing to measure the biological information. Accordingly, first, the measurement timing determination unit 105 refers to the recognition result for the measurement subject and the recognition result for the surrounding conditions of the measurement subject recorded in the recognition result recording unit 104 from before a certain time period to a present time. Then, the measurement timing determination unit 105 determines whether it is a measurement timing using the contents of the latest recognition result and the history of the recognition result up thereto.

For example, the measurement timing determination unit 105 retains a historical pattern of the recognition result and the contents of determination thereto inside as a list. The measurement timing determination unit 105 determines whether it is a measurement timing according to the list every time the recognition result is referred to from the recognition result recording unit 104.

As a more specific example, when a position in the image where the measurement subject is recognized is located at the same place for a certain time period, and a change in the recognition result of the posture of the person to be detected at the place at the same time is not recognized, the measurement timing determination unit 105 determines that it is a measurement timing. Thereafter, the processing proceeds to step S108.

In step S108, the measurement timing determination unit 105 branches the processing according to the determination result in step S107. If the measurement timing determination unit 105 has determined that it is a measurement timing of the biological information in step S107 (YES in step S108), the measurement timing determination unit 105 transmits the instruction to measure the biological information to the measurement unit 106. Then, the measurement timing determination unit 105 advances the processing to step S109. If the measurement timing determination unit 105 has determined that it is not a measurement timing of the biological information in step S107 (NO in step S108), the measurement timing determination unit 105 returns the processing to step S101.

In step S109, the measurement unit 106 measures the biological information. The measurement unit 106 transmits the measurement value of the biological information obtained by measurement to the measurement value correction unit 107. Then, the processing proceeds to step S110.

In step S110, the measurement value correction unit 107 corrects the measurement value of the biological information of the measurement subject received from the measurement unit 106, if needed. Accordingly, first, the measurement value correction unit 107 refers to the recognition result for the measurement subject and the recognition result for the surrounding conditions of the measurement subject recorded in the recognition result recording unit 104 from before a certain time period to the present time.

Then, the measurement value correction unit 107 determines whether correction of the measurement value of the biological information obtained from the measurement unit 106 is needed using the contents of the latest recognition result and the history of the recognition result up thereto. For example, in the measurement value correction unit 107, a historical pattern of the recognition result and the contents of determination thereof (whether correction is needed, and if correction is needed, correction amount at that time) are retained in an individual list.

Then, the measurement value correction unit 107 determines whether correction of the measurement value of the biological information obtained at the point of time is needed according to the list every time the recognition result is referred to the recognition result recording unit 104.

As a more specific example, when the surrounding conditions of the measurement subject having white coat hypertension result in “a doctor is present near the measurement subject”, the measurement value correction unit 107 determines that correction is needed since an increase in blood pressure caused by the white coat hypertension is included in the measurement value of the biological information.

When it is determined that correction is needed, the measurement value correction unit 107 determines a correction value using the recognition result for the measurement subject and the recognition result for the surrounding conditions of the measurement subject to make correction of the measurement value of the biological information. For example, the measurement value correction unit 107 determines a correction value by referring to a correspondence list between the recognition result for the measurement subject and the recognition result for the surrounding conditions of the measurement subject retained inside, and the correction value.

After the processing described above, the measurement value correction unit 107 advances the processing to step S111.

In step S111, the measurement value evaluation unit 108 evaluates the measurement value of the biological information received from the measurement value correction unit 107 to associate a tag that indicates the evaluation result with the measurement value of the biological information. Accordingly, first, the measurement value evaluation unit 108 refers to the recognition result for the measurement subject and the recognition result for the surrounding conditions of the measurement subject recorded in the recognition result recording unit 104 from before a certain time period to the present time.

Then, the measurement value evaluation unit 108 evaluates the measurement value of the biological information received from the measurement value correction unit 107 using the contents of the latest recognition result and the history of the recognition result up thereto to associate a tag (“mild”, “rest” etc.) that indicates the evaluation result with the measurement value of the biological information.

For example, the measurement value evaluation unit 108 retains therein a set of a historical pattern of the recognition result and the measurement value of the biological information, and the contents of evaluation thereof as a list. Then, the measurement value evaluation unit 108 evaluates the measurement value of the biological information according to the list every time the recognition result is referred from the recognition result recording unit 104 to associate it with the tag.

As a more specific example, when the history of the recognition result for the surrounding conditions represents that a smiling friend is present near the measurement subject and a blood pressure is higher in value than that at normal time of the measurement subject, the measurement value evaluation unit 108 associates a “high blood pressure though a peaceful state” tag therewith.

As another example, when the recognition result for the surrounding conditions represents that a fiend with an angry face is present near the measurement subject, and a blood pressure is generally high in value, the measurement value evaluation unit 108 associates a “stress state though a high blood pressure” tag therewith.

After the processing described above, the measurement value evaluation unit 108 advances the processing to step S112.

In step S112, the measurement value evaluation unit 108 transmits the measurement value of the biological information to the biological information recording unit 109 to record it. When the tag is associated with the measurement value of the biological information in step S111, the tag is transmitted to the biological information recording unit 109 together therewith, and is recorded.

When the above processing ends, the measurement value evaluation unit 108 returns the processing to step S101.

Through the above-described processing, the biological information processing apparatus 100 is installed in a space where the measurement subject is acting, and executes processing to measure the biological information while considering the recognition result for the surrounding conditions of the measurement subject. Further, conditions during measurement of the measurement subject and surrounding conditions at that time which may affect on the measurement value of the biological information can be known not from a video itself but from the evaluation result of the measurement value (i.e., the tag that indicates the evaluation result) of the biological information.

In the present exemplary embodiment, each unit illustrated in FIG. 1 has been described as mounted on the biological information processing apparatus 100 as hardware. However, the present exemplary embodiment is not limited to this. For example, the measurement subject recognition unit 102, the surrounding condition recognition unit 103, the measurement timing determination unit 105, the measurement value correction unit 107, the measurement value evaluation unit 108, and the like may also be mounted on the biological information processing apparatus 100 as software. In other words, the biological information processing apparatus 100 including a central processing unit (CPU) and a memory as a hardware configuration may also be configured so as to cause the CPU to execute a program stored in the memory to realize the software.

Next, a second exemplary embodiment will be described. A biological information processing apparatus according to the second exemplary embodiment is attached to the biological information measurement subject, recognizes the surrounding conditions of the subject, and measures the biological information of the subject while considering the recognition result.

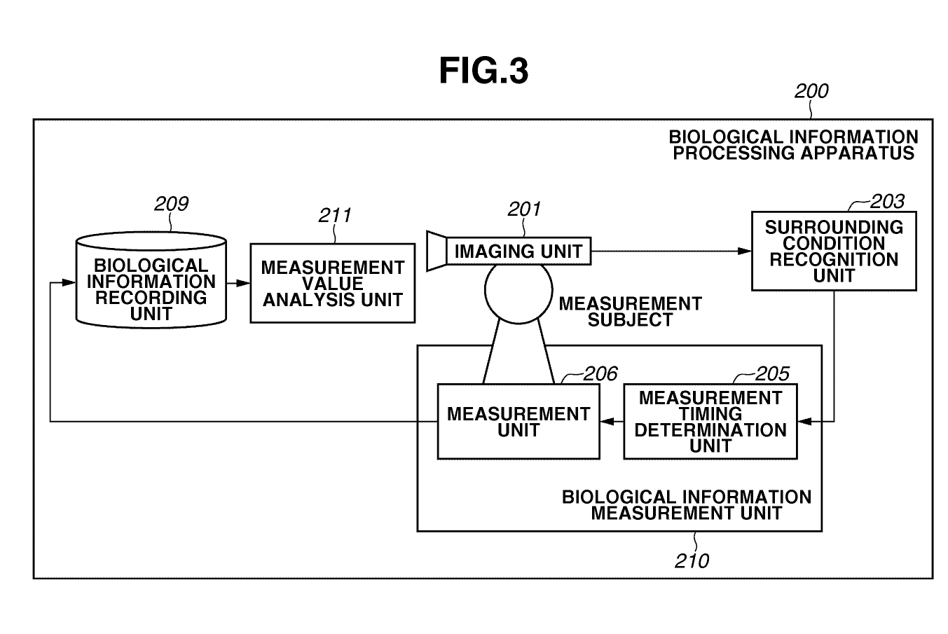

FIG. 3 is a block diagram illustrating an outline of a biological information processing apparatus 200 according to the present exemplary embodiment. As illustrated in FIG. 3, the biological information processing apparatus 200 includes an imaging unit 201, a surrounding condition recognition unit 203, a biological information measurement unit 210, a biological information recording unit 209, and a measurement value analysis unit 211. Similarly, as illustrated in FIG. 3, the biological information measurement unit 210 contained in the biological information processing apparatus 200 includes a measurement timing determination unit 205 and a measurement unit 206.

The imaging unit 201 includes a camera, is attached to the measurement subject, and images an actual space around the measurement subject. The imaging unit 201 may image the visible area of the measurement subject with the imaging unit 201 attached to the head or suspended from the neck. The imaging unit 201 may image the whole circumferences of the measurement subject with the imaging unit 201 attached on the head or the shoulder. The imaging unit 201 outputs the captured video to the surrounding condition recognition unit 103.

A camera parameter such as pan, tilt, and zoom may be fixed or changeable. The imaging unit 201 may also include a sensor (e.g., audio sensor, temperature sensor) to measure a phenomenon in which the conditions of the actual space around the measurement subject are reflected together therewith.

The surrounding condition recognition unit 203 receives a video from the imaging unit 201 to execute video recognition for the surrounding conditions of the measurement subject using a known technique. “Video recognition for surrounding conditions of measurement subject” is to recognize “where the place included in the video is”. Further, “video recognition for surrounding conditions of measurement subject” is also to recognize “who the person included in the video is, what sort of relation the person has to the measurement subject, and what the person intends to do”.

For example, the surrounding condition recognition unit 203 determines “whether the place is located in the hospital” based on whether the image feature of an object (sphygmomanometer, syringe, etc.) that is stored in the surrounding condition recognition unit 203 and is commonly present in the hospital is detected in the video received from the imaging unit 201.

Then, the surrounding condition recognition unit 203 determines “whether a doctor or a nurse is present in the video” based on whether the image feature of a person dressed with a white coat that is stored therein is detected in the video received from the imaging unit 201. Further, the surrounding condition recognition unit 203 may determine that the place is located in the “hospital” and “the person dressed with the white coat is a doctor or a nurse” by simultaneously recognizing the person and the object particular to the hospital such as “a person dressed with a white coat” or “a sphygmomanometer”.

When the imaging unit 201 includes a sensor to measure an phenomenon reflecting the conditions of the actual space around the measurement subject, the surrounding condition recognition unit 203 receives the measurement value of the sensor from the imaging unit 201. Then, the surrounding condition recognition unit 203 may also execute “video recognition for surrounding conditions of measurement subject” using the received measurement value.

For example, when an audio sensor provided on the imaging unit 201 detects a conversation, the surrounding condition recognition unit 203 may recognize voice of the conversation to narrow down the possible recognition result based on the result, whereby the surrounding conditions unable to be specified only by the video may also be specified.

The surrounding condition recognition unit 203 transmits the recognized result to the measurement timing determination unit 205.

The biological information measurement unit 210 executes processing concerning the measurement of the biological information. More specifically, the biological information measurement unit 210 determines a timing to measure the biological information, and executes processing to measure the biological information. The biological information measurement unit 210 transmits the result of various types of executed processing to the biological information recording unit 209.

The measurement timing determination unit 205 determines the timing to measure the biological information of the measurement subject using the recognition result for the surrounding conditions of the measurement subject received from the surrounding condition recognition unit 203. For example, when the recognition result can be interpreted as that “the measurement subject is present in a living room of her/his own house and nobody is present other than the measurement subject”, the measurement timing determination unit 205 determines to measure the biological information at that point of time.

On the other hand, when the recognition result can be interpreted as that “the measurement subject is present out of doors and is moving”, the measurement timing determination unit 205 determines not to measure the biological information at that timing. The measurement timing determination unit 205 determines a measurement timing based on such a recognition result.

For example, the measurement timing determination unit 205 has a pattern of the recognition result and the contents of determination thereto inside thereof as a list. Then, the measurement timing determination unit 205 determines whether the timing is a measurement timing according to the list every time the recognition result is received from the surrounding condition recognition unit 203. When it is determined that the biological information is to be measured, the measurement timing determination unit 205 transmits an instruction to measure the biological information to the measurement unit 206. At that time, the measurement timing determination unit 205 also transmits the recognition result to the measurement unit 206 together therewith.

The measurement unit 206 is a sensor to measure biological information of the measurement subject. For example, the measurement unit 206 includes a thermometer, a sphygmomanometer, a heart rate meter, an electrocardiograph, and the like. The measurement unit 206 may measure the biological information by directly contacting the measurement subject or may measure the biological information without contacting the measurement subject. However, in the present exemplary embodiment, it is assumed that the measurement unit 206 can be carried by the measurement subject and measure the biological information of the measurement subject anytime, anywhere.

The timing at which the measurement unit 206 measures the biological information is determined by the measurement timing determination unit 205. More particularly, if an instruction to measure the biological information is received from the measurement timing determination unit 205, the measurement unit 206 measures the biological information. The measurement unit 206 transmits the measurement value of the biological information to the biological information recording unit 209. At this time, the measurement unit 206 also transmits the recognition result received from the surrounding condition recognition unit 203 to the measurement unit 206 together therewith.

The biological information recording unit 209 receives the measurement value of the biological information from the measurement unit 206 to record this therein. At this time, the biological information recording unit 209 also records the recognition result received from the surrounding condition recognition unit 203 via the measurement unit 206 together therewith. The recorded measurement value of the biological information and the recognition result corresponding thereto are referred to by the measurement value analysis unit 211.

The measurement value analysis unit 211 refers to the history of the measurement value of the biological information and the recognition result corresponding thereto recorded in the biological information recording unit 209 to analyze the individual measurement value of the biological information. For example, in order to analyze a long term fluctuation of the biological information (pulse, etc.) in a certain condition (during rest in living room), the measurement value analysis unit 211 collects only the measurement value of the biological information (pulse rate) corresponding to the recognition result of the surrounding conditions which indicates “during rest in living room” to analyze the time fluctuation thereof.

The result of analysis is disclosed to a doctor or a measurement subject via a system or a display (not illustrated). Then, the doctor or the measurement subject can confirm whether the biological information continuously measured on the same conditions has a fluctuation or a regular fluctuation. Thus, an abnormal cardiac rhythm can be found in early stages.

The above-described is the configuration concerning the biological information processing apparatus 200 according to the present exemplary embodiment.

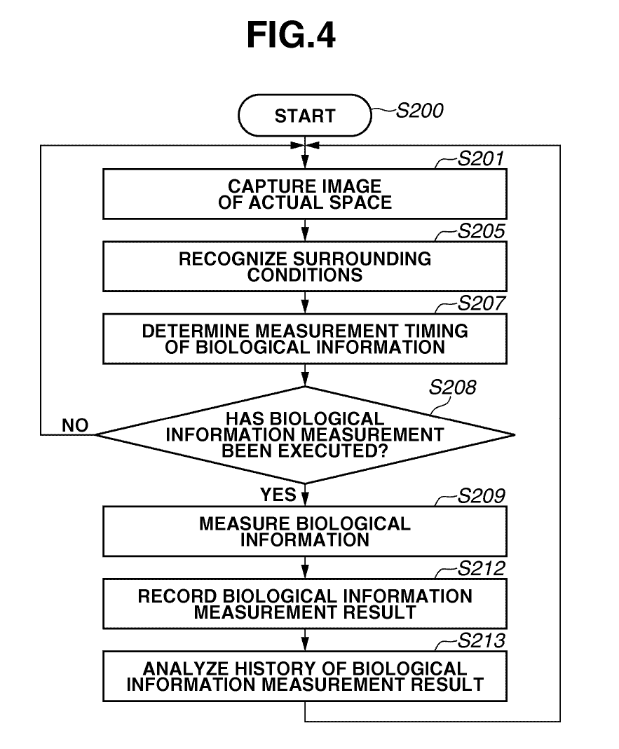

Subsequently, referring to a flowchart illustrated in FIG. 4, the processing to be executed by the biological information processing apparatus 200 according to the present exemplary embodiment will be described.

When the processing is started in step S200, first, the processing proceeds to step S201. In step S201, the imaging unit 201 captures an image of an actual space around the measurement subject. The imaging unit 201 transmits the captured video to the surrounding condition recognition unit 203. Then, the processing proceeds to step S205.

In step S205, the surrounding condition recognition unit 203 executes video recognition processing for the surrounding conditions of the measurement subject to the video transmitted from the imaging unit 201 using a known technique. Through this processing, “where the place included in the video is located” and “who the person included in the video is, what sort of relation the person has to the measurement subject, and what the person intends to do” are recognized. As a result, for example, the recognition result such that “the measurement subject is present in a hospital” and “the measurement subject is present together with a smiling person” is obtained.

Then, the surrounding condition recognition unit 203 advances the processing to step S207.

In step S207, the measurement timing determination unit 205 determines whether the timing is a measurement timing using the contents of the recognition result for the surrounding conditions of the measurement subject. For example, the measurement timing determination unit 205 retains a pattern of the recognition result and the contents of determination thereto inside. Then, the measurement timing determination unit 205 determines whether the timing is measurement timing according to the list every time the recognition result is received from the surrounding condition recognition unit 203.

As a more specific example, when it is recognized that the position recognition result of the measurement subject is located in her/his own house and no person is present around the measurement subject, the measurement timing determination unit 205 determines that the timing is a measurement timing. Thereafter, the measurement timing determination unit 205 advances the processing to step S208.

In step S208, the measurement timing determination unit 205 branches the processing based on the result of determination in step S207. When it is determined that the biological information is to be measured (YES in step S208), the measurement timing determination unit 205 transmits an instruction to measure the biological information and the recognition result generated in step S205 to the measurement unit 206. Then, the processing proceeds to step S209. If it is determined that the biological information is not to be measured (NO in step S208), the processing returns to step S201. In step S209, the measurement unit 206 measures the biological information.

The measurement unit 206 transmits the measurement value of the biological information obtained by measurement to the biological information recording unit 209 together with the recognition result received from the measurement timing determination unit 205. Then, the processing proceeds to step S212.

In step S212, the measurement unit 206 transmits the measurement value of the biological information and the recognition result of the surrounding conditions corresponding thereto to the biological information recording unit 209 to record thereof. Thereafter, the measurement unit 206 advances the processing to step S213.

In step S213, the measurement value analysis unit 211 analyzes the history of the measurement value recorded in the biological information recording unit 209. The measurement value analysis unit 211 executes analysis using the recognition result of the surrounding conditions corresponding to each measurement value of the biological information. The result of analysis is disclosed to a doctor, a measurement subject, or the like via a system, a display, or the like (not illustrated). When the above-described processing ends, the measurement value analysis unit 211 returns the processing to step S201.

Through the above-described processing, the biological information processing apparatus 200 is attached to the measurement subject as the biological information measurement subject, recognizes the surrounding conditions of the measurement subject, and executes processing to measure the biological information of the measurement subject while considering the recognition result. Further, the biological information processing apparatus 200 can analyze the history of the continuously recorded measurement values of the biological information in view of the surrounding conditions when the biological information is measured.

In the present exemplary embodiment, each configuration illustrated in FIG. 3 has been described as that mounted on the biological information processing apparatus 200 as hardware. However, the present exemplary embodiment is not limited to this. For example, the surrounding condition recognition unit 203, the measurement timing determination unit 205, the measurement value analysis unit 211, and the like may also be mounted on the biological information processing apparatus 200 as software.

In other words, the biological information processing apparatus 200 including a CPU and a memory as a hardware configuration may also be configured so as to cause the CPU to execute a program stored in the memory to realize the software.

According to the above-described respective exemplary embodiments, the biological information of the measurement subject can be measured in consideration of a fluctuation caused by the surrounding conditions of the biological information measurement subject.

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures, and functions.

This application claims priority from Japanese Patent Application No. 2010-117665 filed May 21, 2010, which is hereby incorporated by reference herein in its entirety.

What is claimed is:

1. A biological information measurement apparatus comprising:
an input unit configured to input a captured image of a subject whose biological information is to be measured;
a surrounding condition recognition unit configured to recognize surrounding conditions of the subject from the image; and
a control unit configured to control a measurement unit, which measures the biological information of the subject, based on the surrounding conditions recognized by the surrounding condition recognition unit.

2. The biological information measurement apparatus according to claim 1, wherein the surrounding condition recognition unit determines whether other person is present around the subject and recognizes a state of the person if present.

3. The biological information measurement apparatus according to claim 1, wherein the surrounding condition recognition unit recognizes an object present around the subject and a state of the object.

4. The biological information measurement apparatus according to claim 3, wherein the surrounding condition recognition unit recognizes an apparatus present around the subject and an operation state of the apparatus.

5. The biological information measurement apparatus according to claim 1, wherein the control unit determines whether it is a timing to measure the biological information of the subject based on the surrounding conditions recognized by the surrounding condition recognition unit, and instructs the measurement unit to execute measurement when the control unit has determined that it is a timing to measure the biological information of the subject.

6. The biological information measurement apparatus according to claim 1, further comprising:
a correction unit configured to correct a measurement value of the biological information measured by the measurement unit based on the surrounding conditions recognized by the surrounding condition recognition unit.

7. The biological information measurement apparatus according to claim 6, further comprising:
an evaluation unit configured to evaluate the measurement value of the biological information corrected by the correction unit based on the surrounding conditions recognized by the surrounding condition recognition unit.

8. The biological information measurement apparatus according to claim 7, further comprising:
a surrounding condition recording unit configured to record surrounding conditions of the subject recognized by the surrounding condition recognition unit, and
a biological information recording unit configured to record biological information measured by the measurement unit,
wherein the evaluation unit evaluates the measurement value of the corrected biological information based on a history of the surrounding conditions of the subject recorded in the surrounding condition recording unit and a history of the biological information recorded in the biological information recording unit.

9. The biological information measurement apparatus according to claim 6, further comprising:
a surrounding condition recording unit configured to record the surrounding conditions of the subject recognized by the surrounding condition recognition unit, and
a biological information recording unit configured to record the biological information measured by the measurement unit,
wherein the correction unit corrects the measurement value of the biological information based on the history of the surrounding conditions of the subject recorded in the surrounding condition recording unit and the history of the biological information recorded in the biological information recording unit.

10. The biological information measurement apparatus according to claim 1, further comprising:
a surrounding condition recording unit configured to record recognition results of the surrounding condition recognition unit,
wherein the control unit controls the measurement unit so as to measure the biological information of the subject based on the history of the recognition results of the recognition unit recorded in the recording unit.

11. The biological information measurement apparatus according to claim 1, further comprising:
a subject recognition unit configured to recognize a state of the subject from the image,
wherein, when the subject is in a state where the biological information can be measured by the subject recognition unit, the surrounding condition recognition unit recognizes surrounding conditions of the subject.

12. A biological information measurement method comprising:
inputting a captured image of a subject whose biological information is to be measured;
recognizing surrounding conditions of the subject; and
measuring the biological information of the subject based on the recognized surrounding conditions.

13. A non-transitory computer-readable storage medium storing a program to cause a computer to execute the biological information measurement method according to claim 12.
